(19) United States
(12) Patent Application Publication
(10) Pub. No.: US 2015/0246095 A1
(43) Pub. Date: Sep. 3, 2015

(54) METHODS AND PHARMACEUTICAL COMPOSITIONS FOR THE PROPHYLACTIC TREATMENT OF BACTERIAL SUPERINFECTIONS POST-INFLUENZA

(71) Applicants: INSERM (Institut National de la Santé et de la Recherche Médicale), Paris (FR); INSTITUT PASTEUR DE LILLE, Lille (FR); UNIVERSITE DE LILLE I-SCIENCES ET TECHNOLOGIES, Villeneuve-D'ascq (FR); UNIVERSITE DU DROIT ET DE LA SANTE LILLE 2, Lille (FR); CENTRE NATIONAL DE LA RECHERCHE SCIENTIFQUE (CNRS), Paris (FR)

(72) Inventors: Francois Trottein, Lille (FR); Stoyan Ivanov, St. Louis, MO (US); Josette Fontaine, Lille (FR); Christelle Faveeuw, Lille (FR)

(21) Appl. No.: 14/430,405

(22) PCT Filed: Oct. 1, 2013
(86) PCT No.: PCT/EP2013/070440

§ 371 Date: Mar. 23, 2015

(30) Foreign Application Priority Data
Oct. 3, 2012 (EP) 12306203.6

(51) Int. Cl. A61K 38/20; C12Q 1/68; G01N 33/68; A61K 45/06

(52) U.S. Cl. CPC A61K 38/20; A61K 45/06; C12Q 1/6883; G01N 33/6869; C12Q 2600/156; C12Q 2600/158; C12Q 2600/118; G01N 2800/50; G01N 2800/26

(57) ABSTRACT

The present invention relates to methods and pharmaceutical compositions for the prophylactic treatment of bacterial superinfections post-influenza. In particular, the present invention relates to an interleukin 22 (IL-22) polypeptide for use in the prophylactic treatment of bacterial superinfections post-influenza in a subject in need thereof.

### FIELD OF THE INVENTION

The present invention relates to methods and pharmaceutical compositions for the prophylactic treatment of bacterial superinfections post-influenza.

### BACKGROUND OF THE INVENTION

Influenza A virus (IAV) infection is one of the most important causes of respiratory tract diseases and is responsible for widespread morbidity and mortality. During the first several days after infection, the host develops a complex and effective innate immune response that allows to contain IAV replication pending the development of adaptive immune responses. However, at later time points, increased susceptibility to bacterial superinfection can occur leading to mortality during IAV epidemics and pandemics. For instance bacterial pneumonias accounted for the majority of deaths (˜50 million deaths worldwide) in the 1918 pandemic (Spanish flu). Among the predominant bacteria species causing bacterial superinfection post-IAV are Streptococcus pneumoniae (the pneumococcus), Haemophilus influenzae and Staphylococcus aureus. Thus, there is a need for prophylactic treatment of bacterial superinfections post-influenza.

Although there are evidences of specific features of individual types of bacteria, the mechanisms leading to enhanced susceptibility to secondary bacterial infection seem to be broad-based and include alterations of mechanical and immunological defences. Indeed, alteration of the physical barriers to bacterial adhesion and invasion including alteration of the mucosa as well as the exposition of new attachment sites for the bacteria have been described. In parallel, impairment of the host innate (rather than adaptive) response is a cardinal feature of bacterial-associated pneumonia post-influenza challenge.

Interleukin-22, a member of the IL-10 cytokine family, plays a dual role in auto-immune and inflammatory diseases (for reviews, (1, 2)). It is produced by T helper and cytotoxic lymphocytes (Th17/Tc17, Th22/Tc22 in humans) and by cells of the innate immune system. Among the later group, certain subsets of retinoic acid receptor-related orphan receptor-γt (RORγt)-positive γδ T lymphocytes and innate lymphoid cells (ILCs) represent early sources of IL-22 (1, 3-10). More recently, IL-22 was shown to be produced by RORγt-positive invariant NKT (iNKT) lymphocytes, a subset of lipid-reactive “innate-like” T cell population (11-13).

IL-22 solely acts on nonhematopoietic cells, including hepatocytes and epithelial cells, to exert both proinflammatory and tissue-protective properties depending on the context and the tissue in which it is expressed (1, 14-17). In experimental non-infectious systems, IL-22 exerts a potent protective effect on hepatocytes and epithelial cells at barrier surfaces, particularly in the intestine (18-21). In the lungs, IL-22 protects against experimental lung fibrosis induced by chronic exposure with Bacillus subtilis (22) and against ventilator-induced lung injury (23). IL-22 also appears to limit Th2-mediated airway inflammation and tissue damage during asthma (24-26). On the other hand, IL-22 favours dermal inflammation and acanthosis, bleomycin-induced airway inflammation, collagen-induced arthritis and LPS-shock, in part by enhancing tissue inflammation in concert with inflammatory factors (27-30).

During infection, IL-22 production by innate cells or effector conventional T cells plays a dual role depending on the pathogen and the tissue. The early production of IL-22 by innate immune cells is crucial for host protective immunity to extracellular bacteria including Klebsiella pneumoniae in the lung and Citrobacter rodentium in the intestine (31, 32). In this setting, the protective effect of IL-22 is in part due to its effect on the expression of antimicrobial peptides from epithelial cells. IL-22 also displays a role in the clearance of Staphylococcus aureus in the lungs (33). On the other hand, IL-22 has no substantial role in host defence against Mycobacterium tuberculosis, M. avium, Listeria monocytogenes, Candida albicans or Schistosoma mansoni (34-36). IL-22 provides protective innate immunity when the adaptive immune system is impaired. This redundant function has been described during infection with Candida albicans, C. rodentium and Eimeria falciformis (37-39). Finally, a deleterious role for IL-22 on intestinal inflammation was reported after oral infection with Toxoplasma gondii (36, 40).

The potential role of IL-22 during viral infection has recently been addressed in the human system. Of importance, IL-22 (probably derived from CD4⁺ T lymphocytes) might participate in resistance to human immunodeficiency virus infection in subjects who do not seroconvert despite multiple exposures to the virus (41-43). On the other hand, although hepatic IL-22 expression is up-regulated in viral hepatitis, recent evidence indicated that IL-22 lacks direct antiviral activity against hepatitis B and C viruses (44, 45). This is in contrast with interferon (IFN)-γ (46), which shares a structural similarity with IL-22 (47, 48). In the mouse system, very few studies have been devoted to investigate the potential role of IL-22 in anti-viral host defence or in virus-associated inflammation and most studies focused on influenza A virus (IAV). Levillayer and colleagues first reported that IL-22 is a candidate gene for the control of mortality during Theiler's virus-induced acute encephalomyelitis (49). The potential role of IL-22 during experimental IAV infection has been examined using neutralizing anti-IL-22 Abs. Guo et al. (50) showed that IL-22 has no or little role during acute H1N1 IAV infection, as assessed by IAV-associated morbidity and mortality. In parallel, Monticelli and colleagues recently reported that during mild H1N1 IAV infection, IL-22 had no impact on the morbidity, on the decreased lung function and on respiratory tissue remodelling, as evaluated by the hyperproliferative epithelial response (51). IL-22 was recently reported to participate in airway epithelial regeneration during the later (resolution) phase of H1N1 IAV infection (83).

### SUMMARY OF THE INVENTION

The present invention relates to an interleukin 22 (IL-22) polypeptide for use in the prophylactic treatment of bacterial superinfections post-influenza in a subject in need thereof

### DETAILED DESCRIPTION OF THE INVENTION

According to the context and the tissue, interleukin (IL)-22 has redundant, protective or pathogenic functions during auto-immune, inflammatory and infectious diseases. Currently little is known about the cellular sources and the role of IL-22 in host defence and pathology during viral infection, including influenza A virus (IAV) infection. To address this issue, a mouse model of respiratory H3N2 influenza infection was used. The inventors show that IL-22, as well as factors associated with IL-22 production, are expressed in the lung tissue during the early phase of IAV infection. RT-PCR analysis show that RORγt-positive cells are the main IL-22 producers during the early phases of IAV infection and that several subsets of RORγt-positive cells express enhanced Il22 transcripts. Using a mild and a severe model of IAV infection, the inventors show that endogenous IL-22 plays no role in the control of IAV replication and in the development of the IAV-specific CD8⁺ T cell response. In the model of acute and lethal infection, the lack of IL-22 did not accelerate or delay IAV-associated pneumonia and animal death. In stark contrast, during mild IAV infection, Il22^{-/-} mice displayed an enhanced lung injury and lower airway epithelial integrity. Of importance, the protective effect of endogenous IL-22 in pulmonary damages appeared to be instrumental in limiting secondary bacterial infection post-influenza. Indeed, after a secondary challenge with Streptococcus pneumoniae, IAV-experienced Il22^{-/-} animals were more susceptible than wild-type controls in terms of survival rate and bacterial growth. Together, IL-22 plays redundant (severe) and beneficial (mild) roles during influenza infection, a finding that could be exploited at a therapeutic level to better control bacterial superinfection post-influenza.

Accordingly the present invention relates to a interleukin 22 (IL-22) polypeptide for use in the prophylactic treatment of bacterial superinfections post-influenza in a subject in need thereof.

The subject can be human or any other animal (e.g., birds and mammals) susceptible to influenza infection (e.g. domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal or pet. In another embodiment, a subject is a human.

According to the invention the subject has an influenza infection. As used herein, the term “influenza infection” has its general meaning in the art and refers to the disease caused by an infection with an influenza virus. In some embodiments of the invention, influenza infection is associated with Influenza virus A or B. In some embodiments of the invention, influenza infection is associated with Influenza virus A. In some specific embodiments of the invention, influenza infection is cause by influenza virus A that is H1N1, H2N2, H3N2 or H5N1.

The terms “prophylaxis” or “prophylactic use” and “prophylactic treatment” as used herein, refer to any medical or public health procedure whose purpose is to prevent a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease.

The prophylactic methods of the invention are particularly suitable for the prevention of bacterial superinfection post-influenza such as, but not limited to infections of the lower respiratory tract (e.g., pneumonia), middle ear infections (e.g., otitis media) and bacterial sinusitis. The bacterial superinfection may be caused by numerous bacterial pathogens. For example, they may be mediated by at least one organism selected from the group consisting of: Streptococcus pneumoniae; Staphylococcus aureus; Haemophilus influenza, Myoplasma species and Moraxella catarrhalis.

The prophylactic methods of the invention are particularly suitable for subjects who are identified as at high risk for developing a bacterial superinfection post-influenza, including subjects who are at least 50 years old, subjects who reside in chronic care facilities, subjects who have chronic disorders of the pulmonary or cardiovascular system, subjects who required regular medical follow-up or hospitalization during the preceding year because of chronic metabolic diseases (including diabetes mellitus), renal dysfunction, hemoglobinopathies, or immunosuppression (including immunosuppression caused by medications or by human immunodeficiency [HIV] virus); children less than 14 years of age, patients between 6 months and 18 years of age who are receiving long-term aspirin therapy, and women who will be in the second or third trimester of pregnancy during the influenza season. More specifically, it is contemplated that the prophylactic method of the invention are suitable for the prevention of bacterial superinfection post-influenza in subjects older than 1 year old and less than 14 years old (i.e., children); subjects between the ages of 50 and 65, and adults who are older than 65 years of age.

In some embodiments, the subject has an IL-22 deficiency (i.e. a dysregulation of IL-22 or IL-22 receptor production). Said deficiency may be caused by a disease selected from the group consisting of psoriasis, Crohn's disease and allergic diseases.

The term “IL-22 polypeptide” has its general meaning in the art and includes naturally occurring IL-22 and function conservative variants and modified forms thereof. The IL-22 can be from any source, but typically is a mammalian (e.g., human and non-human primate) IL-22, and more particularly a human IL-22. IL-22 consists of 179 amino acids. Dumoutier et al. reported for the first time the cloning of genes of murine and human IL-22 (Dumoutier, et al., JI, 164:1814-1819, 2000; U.S. Pat. Nos. 6,359,117 and 6,274,710).

“Function-conservative variants” are those in which a given amino acid residue in a polypeptide has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, most preferably at least 85%, and even more preferably at least 90%, and which has the same or substantially similar properties or functions as the native or parent protein to which it is compared.

In specific embodiments, it is contemplated that IL-22 polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 45 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes (see e.g., technologies of established by VectraMed, Plainsboro, N.J.). Such linkers may be used in modifying the IL-22-derived proteins described herein for therapeutic delivery.

In another particular embodiment the IL-22 polypeptide is fused a Fc domain of an immunoglobulin. Suitable immunoglobins are IgG, IgM, IgA, IgD, and IgE. IgG and IgA are preferred IgGs are most preferred, e.g. an IgG1. Said Fc domain may be a complete Fc domain or a function-conservative variant thereof. The IL-22 polypeptide of the invention may be linked to the Fc domain by a linker. The linker may consist of about 1 to 100, preferably 1 to 10 amino acid residues.

According to the invention, IL-22 polypeptide may be produced by conventional automated peptide synthesis methods or by recombinant expression. General principles for designing and making proteins are well known to those of skill in the art.

IL-22 polypeptides of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. IL-22 polypeptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art.

As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), WI38, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the recombinant production of the IL-22-derived proteins of the invention, it would be necessary to employ vectors comprising polynucleotide molecules for encoding the IL-22-derived proteins. Methods of preparing such vectors as well as producing host cells transformed with such vectors are well known to those skilled in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect genes. Examples of regulatory sequences include transcriptional promoters, operators, or enhancers, mRNA ribosomal binding sites, and appropriate sequences which control transcription and translation.

The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the peptides or polypeptides of the invention will of course depend upon the specific host cell to be used, and is within the skill of the ordinary artisan.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the protein of interest (i.e., IL-22, a variant and the like). Thus, a promoter nucleotide sequence is operably linked to a given DNA sequence if the promoter nucleotide sequence directs the transcription of the sequence.

Another aspect of the invention relates to a nucleic acid molecule encoding for an IL-22 polypeptide for use in the prophylactic treatment of a bacterial superinfection post-influenza in a subject in need thereof

Typically, said nucleic acid is a DNA or RNA molecule, which may be included in any suitable vector, such as a plasmid, cosmid, episome, artificial chromosome, phage or a viral vector as above described. So, a further object of the invention relates to a vector comprising a nucleic acid encoding for an IL-22 polypeptide for use in the prophylactic treatment of a bacterial superinfection post-influenza.

The present invention relates to a method for preventing bacterial superinfections post-influenza in a subject in need thereof comprising the step of administrating said patient with therapeutically effective amount of an IL-22 polypeptide (or nucleic acid encoding for IL-22 polypeptides).

By a “therapeutically effective amount” is meant a sufficient amount of IL-22 polypeptides (or nucleic acid encoding for a IL-22 polypeptide) to prevent bacterial superinfections post-influenza at a reasonable benefit/risk ratio applicable to any medical treatment.

It will be understood that the total daily usage of the compounds and compositions of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The IL-22 polypeptides of the invention (or the nucleic acid encoding thereof) may be combined with pharmaceutically acceptable excipients, and optionally sustained-release matrices, such as biodegradable polymers, to form phamaceutical compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The IL-22 polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The pharmaceutical compositions may also be administered to the respiratory tract. The respiratory tract includes the upper airways, including the oropharynx and larynx, followed by the lower airways, which include the trachea followed by bifurcations into the bronchi and bronchioli. Pulmonary delivery compositions can be delivered by inhalation by the patient of a dispersion so that the active ingredient within the dispersion can reach the lung where it can, for example, be readily absorbed through the alveolar region directly into blood circulation. Pulmonary delivery can be achieved by different approaches, including the use of nebulized, aerosolized, micellular and dry powder-based formulations; administration by inhalation may be oral and/or nasal. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self contained. Dry powder dispersion devices, for example, deliver drugs that may be readily formulated as dry powders. A pharmaceutical composition of the invention may be stably stored as lyophilized or spray-dried powders by itself or in combination with suitable powder carriers. The delivery of a pharmaceutical composition of the invention for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament. Examples of pharmaceutical devices for aerosol delivery include metered dose inhalers (MDIs), dry powder inhalers (DPIs), and air-jet nebulizers.

The IL-22 polypeptide (or nucleic acid encoding thereof) may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In some embodiment, the IL-22 polypeptide according to the invention is administered to the patient in combination with an anti-bacterial agent, such as antibiotics. Suitable antibiotics that could be coadministered in combination with the IL-22 polypeptide include, but are not limited to, at least one antibiotic selected from the group consisting of: ceftriaxone, cefotaxime, vancomycin, meropenem, cefepime, ceftazidime, cefuroxime, nafcillin, oxacillin, ampicillin, ticarcillin, ticarcillin/clavulinic acid (Timentin), ampicillin/sulbactam (Unasyn), azithromycin, trimethoprim-sulfamethoxazole, clindamycin, ciprofloxacin, levofloxacin, synercid, amoxicillin, amoxicillin/clavulinic acid (Augmentin), cefuroxime,trimethoprim/sulfamethoxazole, azithromycin, clindamycin, dicloxacillin, ciprofloxacin, levofloxacin, cefixime, cefpodoxime, loracarbef, cefadroxil, cefabutin, cefdinir, and cephradine.

A further aspect of the invention relates to a method of testing whether an influenza infected patient is at risk of having a bacterial superinfection post-influenza which comprises the step of analyzing a biological sample from said patient for:
(i) detecting the presence of a mutation in the gene encoding for IL-22 and/or its associated promoter, and/or
(ii) analyzing the expression of the gene encoding for IL-22.

As used herein, the term “biological sample” refers to any sample from a patient such as blood or serum.

Typical techniques for detecting a mutation in the gene encoding for IL-22 may include restriction fragment length polymorphism, hybridisation techniques, DNA sequencing, exonuclease resistance, microsequencing, solid phase extension using ddNTPs, extension in solution using ddNTPs, oligonucleotide assays, methods for detecting single nucleotide polymorphism such as dynamic allele-specific hybridisation, ligation chain reaction, mini-sequencing, DNA “chips”, allele-specific oligonucleotide hybridisation with single or dual-labelled probes merged with PCR or with molecular beacons, and others.

Analyzing the expression of the gene encoding for IL-22 may be assessed by any of a wide variety of well-known methods for detecting expression of a transcribed nucleic acid or translated protein.

In a preferred embodiment, the expression of the gene encoding for IL-22 is assessed by analyzing the expression of mRNA transcript or mRNA precursors, such as nascent RNA, of said gene. Said analysis can be assessed by preparing mRNA/cDNA from cells in a biological sample from a patient, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip(TM) DNA Arrays (AFF YMETRIX). Advantageously, the analysis of the expression level of mRNA transcribed from the gene encoding for IL-22 involves the process of nucleic acid amplification, e.g., by RT-PCR (the experimental embodiment set forth in U.S. Pat. No. 4,683, 202), ligase chain reaction (BARANY, Proc. Natl. Acad. Sci. USA, vol. 88, p: 189-193, 1991), self sustained sequence replication (GUATELLI et al., Proc. Natl. Acad. Sci. USA, vol. 57, p: 1874-1878, 1990), transcriptional amplification system (KWOH et al., 1989, Proc. Natl. Acad. Sci. USA, vol. 86, p: 1173-1177, 1989), Q-Beta Replicase (LIZARDI et al., Biol. Technology, vol. 6, p: 1197, 1988), rolling circle replication (U.S. Pat. No. 5,854, 033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In another preferred embodiment, the expression of the gene encoding for IL-22 is assessed by analyzing the expression of the protein translated from said gene. Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for IL-22. Said analysis can be assessed by a variety of techniques well known from one of skill in the art including, but not limited to, enzyme immunoassay (EIA), radioimmunoassay (RIA), Western blot analysis and enzyme linked immunoabsorbant assay (RIA).

The method of the invention may comprise comparing the level of expression of the gene encoding for IL-22 in a biological sample from a patient with the normal expression level of said gene in a control. A significantly weaker level of expression of said gene in the biological sample of a patient as compared to the normal expression level is an indication that the patient is at risk of a bacterial superinfection post-influenza. The “normal” level of expression of the gene encoding for IL-22 is the level of expression of said gene in a biological sample of a patient (or a group of patients) who does not develop a bacterial superinfection post-influenza. Preferably, said normal level of expression is assessed in a control sample and preferably, the average expression level of said gene in several control samples.

Patients who are considered at risk of having bacterial superinfections post-influenza are then eligible for the prophylactic treatment of the invention (i.e. administration with an IL-22 polypeptide).

The invention will be further illustrated by the following figures and examples. However, these examples and figures should not be interpreted in any way as limiting the scope of the present invention.

### FIGURES

FIG. 1. Role of endogenous IL-22 on bacterial superinfection post-influenza.

A, C57BL/6 WT animals were infected with 50 PFU of IAV Scotland/20/74/H3N2 strain. The lungs were collected 3 and 7 days p.i.. Il22 mRNA copy numbers were determined by quantitative RT-PCR. Data are expressed as fold increased over average gene expression in mock-treated mice. B, WT or Il22^{-/-} mice were infected, or not, with IAV (50 PFU). Seven days later, IAV-infected or naïve animals were challenged with S. pneumoniae (1×10⁴ CFU) and then observed until death (left panel). Percentages of survival are represented (n=15, three independent experiments). Log-rank test for comparisons of Kaplan-Meier survival curves indicated a significant difference in the mortality of Il22^{-/-} compared with WT animals. Right panel, Double-infected WT or Il22^{-/-} mice were sacrificed 24 hrs after S. pneumoniae challenge and the number of CFU was determined in the lungs. Data represent pooled results from two independent experiments (n=9). Significant differences are designated using a one-way ANOVA analysis followed by a Bonferroni post-test. A and B, * p<0.05. C, The number of macrophages and neutrophils in the BALs was determined at 24 hrs after bacterial challenge. D, The concentrations of IFN-γ, CXCL1 and CXCL2 were determined in the BAL fluids 24 hrs after bacterial challenge. C and D, Data represent the mean±SEM (n=3 (Sp) or n=10 (IAV+Sp)).

FIG. 2. Quantification of bacterial and viral loads and of cytokine production in double-infected WT and Il22^{-/-} mice. The lungs of double-infected WT or IL22^{-/-} mice were collected one day after the S. pneumoniae challenge. A, IAV M2 mRNA copy numbers were determined by quantitative RT-PCR. Data are normalized to expression of gapdh and are expressed as fold increased over average gene expression in single-infected WT mice (day 7 post-IAV). B, Ifng, Il17a and Il17f mRNA copy numbers were determined by quantitative RT-PCR. Data are normalized to expression of Gapdh and are expressed as fold increased over average gene expression in naïve mice. Shown is a representative experiment out of two performed (mean±SEM, n=5).

### EXAMPLE

### Materials & Methods

Virus, Bacteria and Mice

The H3N2 IAV strain Scotland/20/74 and S. pneumoniae serotype 1 clinical isolate E1586 sequence type ST304 were described in (58-61). Interleukin-22^{-/-} mice, backcrossed at least 10 times in C57BL/6 (62), as well as WT littermate controls, were bred in the Ludwig Institute (Brussels, Belgium). RORγt-GFP mice were described in (63, 64). Mice (8 to 10-week-old male) were maintained in a biosafety level 2 facility in the Animal Ressource Center at the Pasteur Institute, Lille. All animal work conformed to the Pasteur Institute, Lille animal care and use committee guidelines (agreement number N° AF 16/20090 from the Comité d'Ethique en Expérimentation Animale Nord Pas-De-Calais).

Abs and Reagents

Monoclonal Abs against mouse TCRβ (APC- and V450-conjugated), TCRγδ (PerCP-Cy5.5-conjugated), CD45 (eFluor605NC-, Pacific Blue- or APC-H7-conjugated), NKp46 (PE-conjugated), CD127 (PE-Cy7-conjugated), CD90.2 (Alexa Fluor 700-conjugated), streptavidin (Alexa Fluor 700- or PE-conjugated) and CD4 (APC-H7-conjugated) were purchased from BD Biosciences (Le Pont de Claix, France). Biotin Mouse Lineage Panel was from BD Biosciences. APC-conjugated and PE-conjugated PBS-57-loaded CD1d tetramers were respectively obtained from ProImmune (Oxford, UK) and the NIAID Tetramer Facility (Emory University, Atlanta, Ga.). The monoclonal Ab against mouse IL-22 (clone 3F11) was a kind gift from Dr W. Ouyang (Genentech, San Francisco, Calif.).

IAV Infection and Assessment of Gene Expression by Quantitative RT-PCR

Mice were anesthetized and administered intranasally with 50 μl of PBS containing different dose (100 or 600 plaque forming unit (PFU)) of virus (Scotland/20/74, H3N2). Total RNA from whole lungs or from cells recovered from the bronchoalveolar lavages (BAL) of mock-treated or IAV-infected mice was extracted and cDNA was synthesized by classical procedures. Quantitative RT-PCR was carried out as described (14). Primers specific for gapdh, Ifng, Il17A, m×1, Ifnb, Il22 and IAV M2 gene were described in (14). ΔCt values were obtained by deducting the raw cycle threshold (Ct values) obtained for gapdh mRNA, the internal standard, from the Ct values obtained for investigated genes.

Analysis of IL-22 Transcript Levels in RORγt Positive Cells During IAV Infection

RORγt-GFP mice were infected, or not, with IAV and lung MNCs were prepared 60 hrs p.i.. RORγt-positive αβ T lymphocytes (CD45⁺ TCRβ⁺), γδ T lymphocytes (CD45³⁰ TCRγδ⁺) and TCRαβ⁻ TCRγδ⁻ (CD45⁺TCRβ⁻ TCRγδ⁻) cells were sorted from naïve and IAV-infected mice. The expression of IL-22 transcript was performed by quantitative RT-PCR.

Analysis of IL-22-Producing Cells

Analysis of IL-22-producing cells was assessed 2 days post-IAV infection. As a possible control, lung MNCs were cultured at 1×10⁷ cells/ml in complete medium containing 10 ng/ml of recombinant mouse IL-1β and IL-23 plus 10 μg/ml Brefeldin A (Sigma-Aldrich, Steinheim, Germany) at 37° C. for 4 hrs. After activation, cells were washed and stained with LIVE/DEAD® Fixable Dead Cell Stain Kit (Life Technologies, Carlsbad, USA) in PBS for 30 min. The cells were washed and incubated with appropriate dilutions of eFluor605NC-conjugated CD45, PE-conjugated PBS-57-loaded CD1d tetramer, V450-conjugated anti-TCRβ Ab and PerCP-Cy5.5-conjugated anti-TCRγδ Ab for 30 min in PBS containing 2% FCS. Cells were washed, and fixed using IC Fixation Buffer (eBioscience, CliniSciences, Montrouge, France). Fixed cells were then permeabilized in Permeabilization Buffer (eBioscience), according to the manufacturer's instructions. Cells were stained with APC-conjugated mAb against IL-22 or control mouse IgG2a mAb and analysed on a LSR Fortessa (BD Biosciences). To analyze the proportions of ILCs in RORγt-positive TCRαβ⁻ TCRγδ⁻ cells, lung MNCs from RORγt-GFP mice were labelled with appropriate dilutions of APC-H7-conjugated anti-CD45, Percp-Cy5.5-conjugated anti-TCRγδ, V450-conjugated anti-TCRβ, a biotin lineage-specific Ab cocktail (TER119, CD11b, Gr1, B220, CD3, CD11c, NK1.1) plus a PE-conjugated streptavidin, PE-Cy7-conjugated anti-CD 127 and AlexaFluor700-conjugated CD90.2. Cells were analysed on a LSR Fortessa.

Assessment of the Mortality Rate and of the Pathology

After IAV infection (600 or 50 PFU), mice were monitored daily for illness and mortality for a period of 17 days. Disease was assessed by measuring lung inflammation, viral load in the lungs, and lethality. Mice found to be moribund were euthanized and considered to have died on that day. Mice were also sacrificed at day 4 p.i. to recover the whole lung and the BALs. For histopathologic examination, lungs were fixed by inflation and immersion in PBS 3.2% paraformaldehyde and embedded in paraffin. To evaluate airway inflammation, we subjected fixed lung slices (5 μm sections) to hematoxylin and eosin (H&E) staining Evaluators who were blinded to genotype scored lung sections (0 [none]-3 [extreme]) according to criteria described in (65).

Analysis of the Viral Load and of the IAV-Specific CD8⁺ T Cell Response

Lungs were homogenized and virus titers determined using a standard plaque assay on Mardin-Darby canine kidney cells. Ifnb and M×1 mRNA expression levels were determined by quantitative RT-PCR as described (65). The number of IAV-specific CD8⁺ T cells was determined as reported (65). For this, cells specific for an immunodominant D^{b}-restricted CD8⁺ T epitope derived from the viral polymerase 2 protein (PA₂₂₄₋₂₃₃) (66) were analyzed. Briefly, lung MNCs were incubated with appropriate dilutions of APC-conjugated anti-CD19, FITC-labelled anti-CD8 and PE-conjugated Pro5® MHC pentamer H-2D^{b} SSLENFRAYV. To assess the functionality of virus-specific CD8⁺ T cells, lung cells were stimulated with the peptide SSLENFRAYV (10 μg/ml ) and cytokine production was assessed by ELISA.

Infection with S. pneumoniae

Mice, infected or not with IAV (50 PFU) 7 days earlier, were intranasally inoculated with 1×10⁴ S. pneumoniae serotype 1. Mice were monitored daily for illness and mortality for a period of 13 days. The number of viable bacteria in the lungs was determined 24 hrs post S. pneumoniae challenge. This was measured by plating lung homogenates onto blood agar plates (67). Colony forming units (CFU) were enumerated 24 hrs later. A morphology-based differential cell count was conducted on cytospin preparations from the BAL fluid and stained with Diff-Quik solution (Sigma).

Statistical Analysis

Results are expressed as the mean±SD or ±SEM. The statistical significance of differences between experimental groups was calculated by a one-way analysis of variance followed by a Bonferroni posttest (GraphPad Prism 4 Software, San Diego, USA). The possibility to use these parametric tests was assessed by checking if the population is Gaussian and the variance is equal (Bartlett's test). Results with a p value of less than 0.05 were considered significant, * p<0.05, ** p<0.01, *** p<0.001.

Results:

IL-22 is Produced in the Lungs During the Early Stages of IAV Infection

IL-22 expression during early IAV infection is ill-defined although a recent report suggested that NK cells might be a primary source two days after infection (56). Relative to mock-treated animals, a higher level of Il22 gene transcript was detected in the lung tissue of IAV-infected mice two and four days p.i. (˜10-fold enhancement). Cells from the alveolar spaces also expressed higher amounts of Il22 messengers at these time points. An enhanced concentration of IL-22 protein was also detected in the lung tissue and alveolar spaces, but only two days p.i. The Th17-derived cytokine IL-22 shares similarities with IL-17. Il17a and IL17f gene transcripts were also found to be up-regulated two and four days p.i. in the BAL cells, but not in pulmonary cells, whereas Il21, another member of the IL-17 family, was not modulated. Augmented IL-17A protein was also detected at day 2 p.i., but only in the BAL fluids.

Interleukin-23, IL-1β, IL-6 and TNF-α have been described to participate in early IL-22 production in some settings (for reviews, (1, 8, 9). Il23, Il1b Il6 and Tnfa gene transcripts were strongly up-regulated in the BAL cells and in the lungs, two and four days p.i.. Enhanced IL-23, IL-1β, IL-6 and TNF-α protein levels were also evidenced at these time points. Of note, RORγt, and to a lesser extent, aryl receptor (AhR), transcription factors known to be crucial in IL-22 synthesis (1, 8, 9), were also up-regulated at the transcript level. Collectively, IL-22 as well as factors known to regulate its expression, are produced in the lungs during the early stages of H3N2 IAV infection.

RORγt-Expressing Cells Produce Enhanced IL-22 Transcripts Early After IAV Infection

We took advantage of RORγt-GFP mice to analyze the early source(s) of IL-22 during IAV infection. RORγt-positive cells purified from IAV-infected mice express a dramatically enhanced level of Il22 gene transcripts compared to RORγt positive cells isolated from non-infected animals. In contrast, influenza infection did not trigger Il22 messenger expression in RORγt negative cells. Of note, NKp46⁺ cells did not express RORγt in the lung tissue and failed to produce Il22 messenger in response to IAV.

The different pulmonary RORγt-expressing cell populations were next sorted from mock-treated and IAV-infected animals and analyzed for Il22 gene transcript expression. In agreement with (64, 68), αβ T lymphocytes and γδ T lymphocytes represented the two major populations expressing RORγt. Invariant NKT cells were discarded from the αβ T lymphocyte pool and were analyzed separately. Another minor population of RORγt⁺ cells was also identified. This population contains −70% of Lin⁻ CD127⁺ CD90⁺ CD4⁻ cells and was thus termed as ILC-enriched population. αβ T lymphocytes, γδ T lymphocytes, iNKT cells, and to a lesser extent the ILC-enriched population, produced a higher level of Il22 mRNAs in the context of IAV infection.

Analysis of IL-22 protein expression by RORγt-expressing cells was next assessed in response to a cocktail of IL-1β and IL-23, used here as a positive control. αβ T lymphocytes (mainly CD4^{neg}), γδ T lymphocytes, iNKT cells and, to a lesser extent cells within the ILC-enriched population, produced IL-22 protein. Of note, lung NKp46⁺ cells failed to express IL-22 in response to IL-1β and IL-23. On the other hand, in the context of IAV infection, IL-22 protein expression was not evidenced by intracellular FACS staining, whatever the cell population analyzed.

IL-22 Deficiency has no Impact on Mouse Survival, Viral Clearance and IAV-Specific CD8⁺ T Cell Response

Severe lung immunopathology strongly contributes to influenza-related morbidity and mortality (69). It is known that IL-22 is a versatile controller of immunopathology (1, 8, 9) but little is known about its role during IAV infection. To address this point, WT and Il22^{-/-} mice were used in survival studies. To rule out potential bias due to genetic background, WT and Il22^{-/-} littermates were used. Upon a lethal dose (600 PFU) of IAV, WT animals demonstrated severe sickness ending in death at day 13. Although not significant, 10-15% of IL22^{-/-} mice survived to the infection out to day 18 p.i. On the contrary, using a sublethal dose (50 PFU), IL22^{-/-} mice displayed a slight, although not significant, decreased resistance compared to WT animals.

To determine whether endogenous IL-22 plays part in viral clearance, the viral load was monitored at day 4 and day 7 p.i. by plaque assay. Whatever the dose of IAV used, Il22^{-/-} mice controlled virus replication in their lungs with kinetics similar to WT animals. In agreement with this, the transcript levels of genes associated with viral replication (ifnb, m×1) were not significantly different between infected WT and Il22^{-/-} mice.

Interleukin-22 has been shown to attenuate Ag-induced pulmonary immune responses in some settings (28, 30-32). We thus compared the virus-specific CD8⁺ T cell response in IAV-infected WT and Il22^{-/-} mice in terms of cell number and cytokine production. Whatever the dose of IAV, the number of lung CD8⁺ D^{b}PA₂₂₄₋₂₃₃⁺ cells were not different between WT and Il22^{-/-} mice, 4 and 7 days p.i. Similarly, upon restimulation with PA₂₂₄₋₂₃₃, the amount of IFN-y released by lung cells was identical. Of note, whilst lung MNCs from IAV-infected mice spontaneously released IL-22 at day 7, IL-22 production was not amplified after IAV peptide restimulation, thus indicating the lack of IAV-specific IL-22-producing CD8⁺ T cells in the context of early IAV, at least in response to the immunodominant peptide used. The virus-specific CD8⁺ T cell response was also similar in lung-draining lymph nodes from WT and Il22^{-/-} mice.

Endogenous IL-22 Plays a Positive Role in the Control of Mild, but not Severe, IAV-Associated Pneumonia

We next investigated the role of endogenous IL-22 on IAV-associated pulmonary pathology. To do so, lungs from WT and Il22^{-/-} mice infected with a lethal or a sublethal dose of IAV were harvested for histology during the acute phase of the pulmonary inflammatory response and of epithelial damages. In conditions where IAV causes acute pneumonia ending by the death of the animals (600 PFU), the lack of IL-22 did not significantly modulate the intensity of the pulmonary inflammation. Indeed, the cellular infiltration in the lungs as well as alveolitis and bronchiolitis scores were not significantly different between infected WT and Il22^{-/-} mice. In both infected animal groups, bronchial epithelia were strongly damaged and this was associated with an impaired epithelial integrity as measured by the high total protein concentration in the BAL fluid.

In stark contrast, upon a sublethal challenge (50 PFU), a significant enhancement of airway inflammation was noticed in Il22^{-/-} mice relative to WT mice as reflected by the higher histopathology scores in the former group. In Il22^{-/-} mice, alveolitis and bronchiolitis were more pronounced relative to WT littermates and an enhanced influx of neutrophils and macrophages was observed in the lung tissue. In addition, epithelia surrounding the bronchi from Il22^{-/-} mice were more damaged. Signs of severe injury, characterized by augmented loss of intercellular cohesion and denuded epithelium were observed in Il22^{-/-} animals. This apparent loss of epithelial integrity was however not associated with a significant enhancement of protein concentration in the BALs. In contrast, the number of red blood cells in the luminal side of the alveoli was dramatically enhanced in Il22^{-/-} mice indicative of alveolar leakage and lesional edema.

IL-22 Deficiency Leads to an Enhanced Susceptibility to Secondary Bacterial Infection Post-Influenza

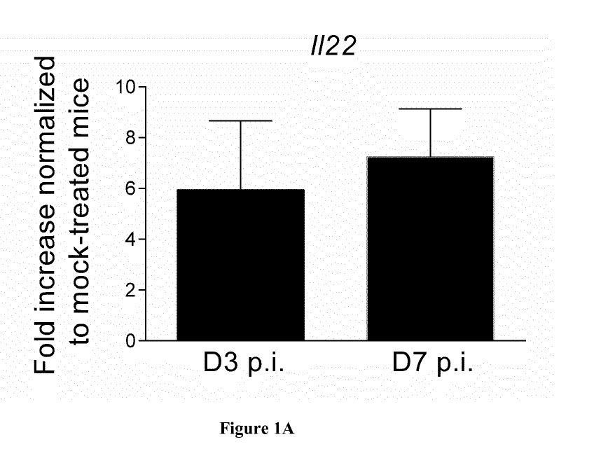

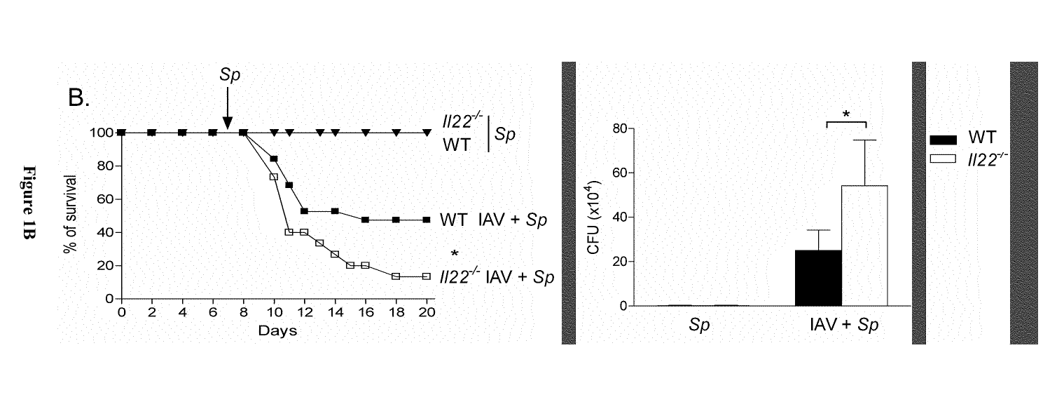

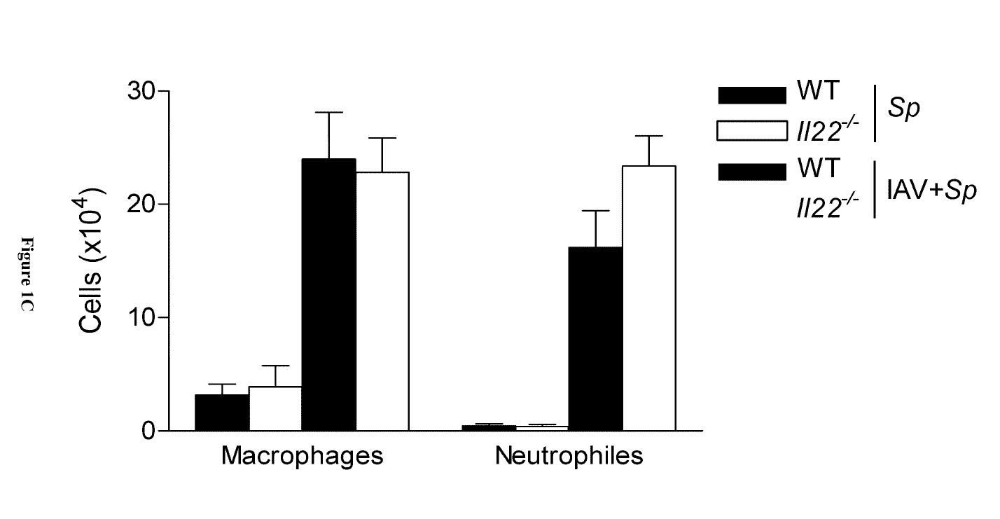

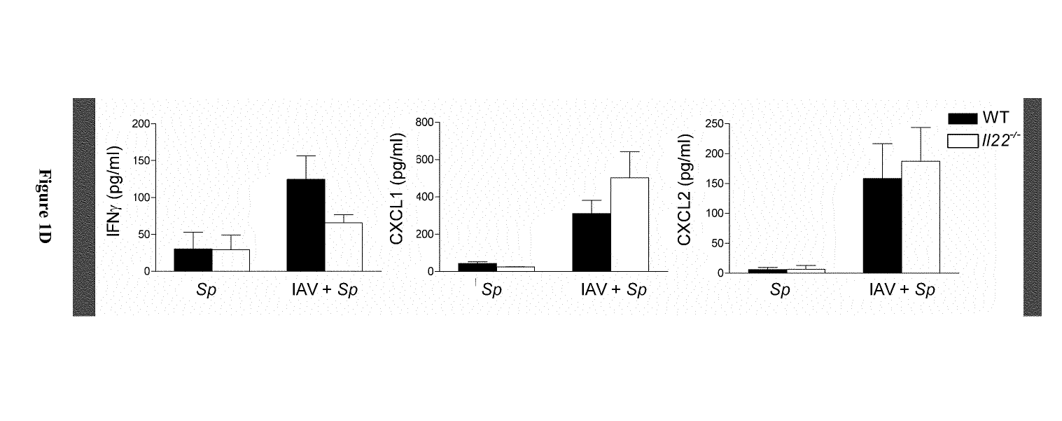

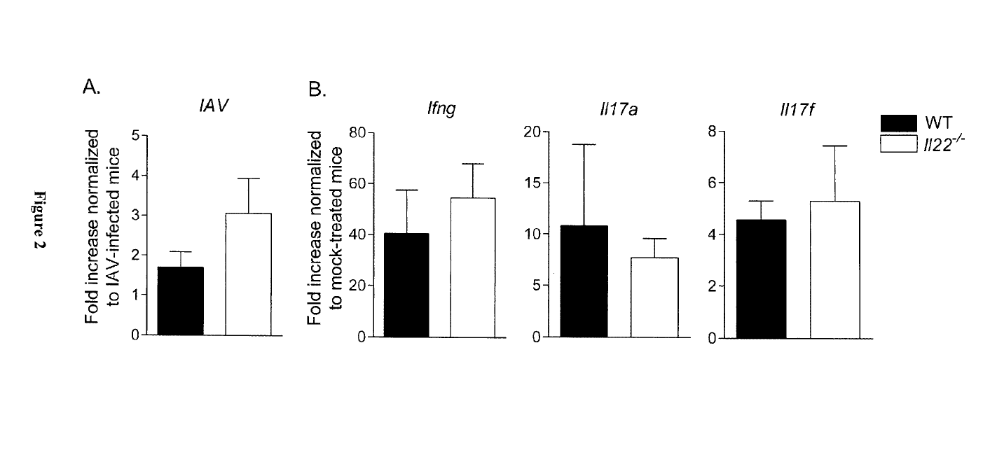

Epithelial damages post-influenza has been proposed to play a part in secondary bacterial infections such as S. pneumoniae (70-76). We thus investigated the role of endogenous IL-22 in bacterial superinfection post-influenza. To this end, WT and Il22^{-/-} mice were infected with a sublethal dose of IAV and, 7 days later, mice were challenged with S. pneumoniae. We chosen this time point since it corresponded to the peak of bacterial susceptibility post-inflenza (not shown). Of note, at the dose of IAV used (50 PFU), Il22 mRNA was detected in the lung tissue at day 3 and 7 p.i. (FIG. 1A). As expected, preceding IAV infection induced animal death after S. pneumoniae challenge (IAV+Sp), whilst in naive mice, and at the dose used, S. pneumoniae challenge did not affect the survival rate (Sp) (FIG. 1B, left panel). Remarkably, whilst 50% of IAV-experienced WT mice succumbed post-S. pneumoniae infection, almost all co-infected Il22^{-/-} mice died from bacterial infection. This effect was associated with an increased number of viable bacteria in the lung tissue (FIG. 1B, right panel). Of importance, the observed effect was probably not due to a direct anti-bacterial effect of endogenous IL-22 per se. Indeed, both non-IAV experienced WT and Il22^{-/-} mice survived and equally cleared bacteria in the lungs after sublethal S. pneumoniae challenge (FIG. 1B).

Thus, the lack of IL-22 worsens the outcome of secondary S. pneumoniae pneumonia. This effect was not associated with a significant higher IAV burden in the lungs (FIG. 2A) nor to a differential recruitment of macrophages and neutrophils in the BALs of double infected mice, cells known to be crucial in the clearance of S. pneumoniae (77, 78) (FIG. 1C). Furthermore, production of factors known to exert anti-pneumococcal effects, including IFN-γ, IL-17 and the neutrophil-attracting factors CXCL1 and CXCL2, were not different between WT and Il22^{-/-} double-infected mice (FIG. 1D and FIG. 2B). Collectively, endogenous IL-22 limits bacterial superinfection post-influenza, a phenomenon probably independent from enhanced host defence mechanisms.

Conclusion:

In the current study, we report that IAV-infected Il22^{-/-} mice have decreased survival and clearance of secondary S. pneumonia infection from the lungs, as compared with similarly infected WT animals. We favour the hypothesis that the protective role of IL-22 in bacterial superinfection post-IAV is rather due to its positive early effect on lung, including epithelial, injury due to IAV infection rather than to an anti-bacterial effector mechanisms per se. Indeed, molecular (IL-17, IFN-γ, CXCL1/2) and cellular (macrophages, neutrophils) effectors associated with early innate defence mechanisms against S. pneumonia were not significantly decreased in the absence of IL-22. Furthermore, Il22^{-/-} mice without antecedent IAV infection controlled S. pneumoniae, at least after a non-lethal challenge. The mechanisms through which IL-22 protects against bacterial secondary infection post-IAV deserve future investigations. In parallel, since in humans, dysregulation of IL-22 or IL-22 receptor production has been reported in certain pathologies including psoriasis, Crohn's disease and allergic diseases, it might be interesting to determine the potential correlation, if any, between susceptibility to bacterial superinfection and IL-22 or IL-22 receptor production in patients. Recent findings suggest that, in combination with antibiotic and antiviral therapies, treatments that protect lung epithelium and/or stimulate lung repair responses could be beneficial in improving survival in patients during influenza and bacterial coinfection (57, 76). In conclusion, supplementation of IL-22 might represent a good option for the prophylactic treatment of bacterial superinfections post-influenza.

### REFERENCES

Throughout this application, various references describe the state of the art to which this invention pertains. The disclosures of these references are hereby incorporated by reference into the present disclosure.
1. Sonnenberg, G. F., L. A. Fouser, and D. Artis. Border patrol: regulation of immunity, inflammation and tissue homeostasis at barrier surfaces by IL-22. Nat Immunol 12:383-390.
2. Ouyang, W., S. Rutz, N. K. Crellin, P. A. Valdez, and S. G. Hymowitz. Regulation and functions of the IL-10 family of cytokines in inflammation and disease. Annu Rev Immunol 29:71-109.
3. Colonna, M. 2009. Interleukin-22-producing natural killer cells and lymphoid tissue inducer-like cells in mucosal immunity. Immunity 31:15-23.
4. Eyerich, S., K. Eyerich, D. Pennino, T. Carbone, F. Nasorri, S. Pallotta, F. Cianfarani, T. Odorisio, C. Traidl-Hoffmann, H. Behrendt, S. R. Durham, C. B. Schmidt-Weber, and A. Cavani. 2009. Th22 cells represent a distinct human T cell subset involved in epidermal immunity and remodeling. J Clin Invest 119:3573-3585.
5. Liang, S. C., X. Y. Tan, D. P. Luxenberg, R. Karim, K. Dunussi-Joannopoulos, M. Collins, and L. A. Fouser. 2006. Interleukin (IL)-22 and IL-17 are coexpressed by Th17 cells and cooperatively enhance expression of antimicrobial peptides. J Exp Med 203:2271-2279.
6. Martin, B., K. Hirota, D. J. Cua, B. Stockinger, and M. Veldhoen. 2009. Interleukin-17-producing gammadelta T cells selectively expand in response to pathogen products and environmental signals. Immunity 31:321-330.
7. Sutton, C. E., S. J. Lalor, C. M. Sweeney, C. F. Brereton, E. C. Lavelle, and K. H. Mills. 2009. Interleukin-1 and IL-23 induce innate IL-17 production from gammadelta T cells, amplifying Th17 responses and autoimmunity. Immunity 31:331-341.
8. Spits, H., and J. P. Di Santo. The expanding family of innate lymphoid cells: regulators and effectors of immunity and tissue remodeling. Nat Immunol 12:21-27.
9. Spits, H., and T. Cupedo. Innate lymphoid cells: emerging insights in development, lineage relationships, and function. Annu Rev Immunol 30:647-675.
10. Dumoutier, L., M. de Heusch, C. Orabona, N. Satoh-Takayama, G. Eberl, J. C. Sirard, J. P. Di Santo, and J. C. Renauld. IL-22 is produced by gammaC-independent CD25+ CCR6+ innate murine spleen cells upon inflammatory stimuli and contributes to LPS-induced lethality. Eur J Immunol 41:1075-1085.
11. Van Maele, L., C. Carnoy, D. Cayet, P. Songhet, L. Dumoutier, I. Ferrero, L. Janot, F. Erard, J. Bertout, H. Leger, F. Sebbane, A. Benecke, J. C. Renauld, W. D. Hardt, B. Ryffel, and J. C. Sirard. TLR5 signaling stimulates the innate production of IL-17 and IL-22 by CD3(neg)CD127+ immune cells in spleen and mucosa. J Immunol 185:1177-1185.
12. Wahl, C., U. M. Wegenka, F. Leithauser, R. Schirmbeck, and J. Reimann. 2009. IL-22-dependent attenuation of T cell-dependent (ConA) hepatitis in herpes virus entry mediator deficiency. J Immunol 182:4521-4528.
13. Doisne, J. M., V. Soulard, C. Becourt, L. Amniai, P. Henrot, C. Havenar-Daughton, C. Blanchet, L. Zitvogel, B. Ryffel, J. M. Cavaillon, J. C. Marie, I. Couillin, and K. Benlagha. Cutting edge: crucial role of IL-1 and IL-23 in the innate IL-17 response of peripheral lymph node NK1.1- invariant NKT cells to bacteria. J Immunol 186:662-666.
14. Paget, C., S. Ivanov, J. Fontaine, J. Renneson, F. Blanc, M. Pichavant, L. Dumoutier, B. Ryffel, J. C. Renauld, P. Gosset, P. Gosset, M. Si-Tahar, C. Faveeuw, and F. Trottein. Interleukin-22 Is Produced by Invariant Natural Killer T Lymphocytes during Influenza A Virus Infection: POTENTIAL ROLE IN PROTECTION AGAINST LUNG EPITHELIAL DAMAGES. J Biol Chem 287:8816-8829.
15. Goto, M., M. Murakawa, K. Kadoshima-Yamaoka, Y. Tanaka, K. Nagahira, Y. Fukuda, and T. Nishimura. 2009. Murine NKT cells produce Th17 cytokine interleukin-22. Cell Immunol 254:81-84.
16. Moreira-Teixeira, L., M. Resende, M. Coffre, O. Devergne, J. P. Herbeuval, O. Hermine, E. Schneider, L. Rogge, F. M. Ruemmele, M. Dy, A. Cordeiro-da-Silva, and M. C. Leite-de-Moraes. Proinflammatory environment dictates the IL-17-producing capacity of human invariant NKT cells. J Immunol 186:5758-5765.
17. Kumar, P., M. S. Thakar, W. Ouyang, and S. Malarkannan. IL-22 from conventional NK cells is epithelial regenerative and inflammation protective during influenza infection. Mucosal Immunol.
18. Renauld, J. C. 2003. Class II cytokine receptors and their ligands: key antiviral and inflammatory modulators. Nat Rev Immunol 3:667-676.
19. Wolk, K., S. Kunz, E. Witte, M. Friedrich, K. Asadullah, and R. Sabat. 2004. IL-22 increases the innate immunity of tissues. Immunity 21:241-254.
20. Witte, E., K. Witte, K. Warszawska, R. Sabat, and K. Wolk. Interleukin-22: a cytokine produced by T, NK and NKT cell subsets, with importance in the innate immune defense and tissue protection. Cytokine Growth Factor Rev 21:365-379.
21. Eyerich, S., K. Eyerich, A. Cavani, and C. Schmidt-Weber. IL-17 and IL-22: siblings, not twins. Trends Immunol 31:354-361.
22. Zenewicz, L. A., G. D. Yancopoulos, D. M. Valenzuela, A. J. Murphy, M. Karow, and R. A. Flavell. 2007. Interleukin-22 but not interleukin-17 provides protection to hepatocytes during acute liver inflammation. Immunity 27:647-659.
23. Pickert, G., C. Neufert, M. Leppkes, Y. Zheng, N. Wittkopf, M. Warntjen, H. A. Lehr, S. Hirth, B. Weigmann, S. Wirtz, W. Ouyang, M. F. Neurath, and C. Becker. 2009. STAT3 links IL-22 signaling in intestinal epithelial cells to mucosal wound healing. J Exp Med 206:1465-1472.
24. Radaeva, S., R. Sun, H. N. Pan, F. Hong, and B. Gao. 2004. Interleukin 22 (IL-22) plays a protective role in T cell-mediated murine hepatitis: IL-22 is a survival factor for hepatocytes via STAT3 activation. Hepatology 39:1332-1342.
25. Sugimoto, K., A. Ogawa, E. Mizoguchi, Y. Shimomura, A. Andoh, A. K. Bhan, R. S. Blumberg, R. J. Xavier, and A. Mizoguchi. 2008. IL-22 ameliorates intestinal inflammation in a mouse model of ulcerative colitis. J Clin Invest 118:534-544.
26. Dudakov, J. A., A. M. Hanash, R. R. Jenq, L. F. Young, A. Ghosh, N. V. Singer, M. L. West, O. M. Smith, A. M. Holland, J. J. Tsai, R. L. Boyd, and M. R. van den Brink. Interleukin-22 drives endogenous thymic regeneration in mice. Science 336:91-95.
27. Sonnenberg, G. F., L. A. Monticelli, T. Alenghat, T. C. Fung, N. A. Hutnick, J. Kunisawa, N. Shibata, S. Grunberg, R. Sinha, A. M. Zahm, M. R. Tardif, T. Sathaliyawala, M. Kubota, D. L. Farber, R. G. Collman, A. Shaked, L. A. Fouser, D. B. Weiner, P. A. Tessier, J. R. Friedman, H. Kiyono, F. D. Bushman, K. M. Chang, and D. Artis. Innate lymphoid cells promote anatomical containment of lymphoid-resident commensal bacteria. Science 336:1321-1325.
28. Simonian, P. L., F. Wehrmann, C. L. Roark, W. K. Born, R. L. O'Brien, and A. P. Fontenot. gammadelta T cells protect against lung fibrosis via IL-22. J Exp Med 207:2239-2253.
29. Hoegl, S., M. Bachmann, P. Scheiermann, I. Goren, C. Hofstetter, J. Pfeilschifter, B. Zwissler, and H. Muhl. Protective properties of inhaled IL-22 in a model of ventilator-induced lung injury. Am J Respir Cell Mol Biol 44:369-376.
30. Takahashi, K., K. Hirose, S. Kawashima, Y. Niwa, H. Wakashin, A. Iwata, K. Tokoyoda, J. C. Renauld, I. Iwamoto, T. Nakayama, and H. Nakajima. IL-22 attenuates IL-25 production by lung epithelial cells and inhibits antigen-induced eosinophilic airway inflammation. J Allergy Clin Immunol 128:1067-1076 e1061-1066.
31. Besnard, A. G., R. Sabat, L. Dumoutier, J. C. Renauld, M. Willart, B. Lambrecht, M. M. Teixeira, S. Charron, L. Fick, F. Erard, K. Warszawska, K. Wolk, V. Quesniaux, B. Ryffel, and D. Togbe. Dual Role of IL-22 in allergic airway inflammation and its cross-talk with IL-17A. Am J Respir Crit Care Med 183:1153-1163.
32. Nakagome, K., M. Imamura, K. Kawahata, H. Harada, K. Okunishi, T. Matsumoto, O. Sasaki, R. Tanaka, M. R. Kano, H. Chang, H. Hanawa, J. Miyazaki, K. Yamamoto, and M. Dohi. High expression of IL-22 suppresses antigen-induced immune responses and eosinophilic airway inflammation via an IL-10-associated mechanism. J Immunol 187:5077-5089.
33. Geboes, L., L. Dumoutier, H. Kelchtermans, E. Schurgers, T. Mitera, J. C. Renauld, and P. Matthys. 2009. Proinflammatory role of the Th17 cytokine interleukin-22 in collagen-induced arthritis in C57BL/6 mice. Arthritis Rheum 60:390-395.
34. Sonnenberg, G. F., M. G. Nair, T. J. Kim, C. Zaph, L. A. Fouser, and D. Artis. Pathological versus protective functions of IL-22 in airway inflammation are regulated by IL-17A. J Exp Med 207:1293-1305.
35. Zheng, Y., D. M. Danilenko, P. Valdez, I. Kasman, J. Eastham-Anderson, J. Wu, and W. Ouyang. 2007. Interleukin-22, a T(H)17 cytokine, mediates IL-23-induced dermal inflammation and acanthosis. Nature 445:648-651.
36. Van Belle, A. B., M. de Heusch, M. M. Lemaire, E. Hendrickx, G. Warnier, K. Dunussi-Joannopoulos, L. A. Fouser, J. C. Renauld, and L. Dumoutier. IL-22 is required for imiquimod-induced psoriasiform skin inflammation in mice. J Immunol 188:462-469.
37. Aujla, S. J., Y. R. Chan, M. Zheng, M. Fei, D. J. Askew, D. A. Pociask, T. A. Reinhart, F. McAllister, J. Edeal, K. Gaus, S. Husain, J. L. Kreindler, P. J. Dubin, J. M. Pilewski, M. M. Myerburg, C. A. Mason, Y. Iwakura, and J. K. Kolls. 2008. IL-22 mediates mucosal host defense against Gram-negative bacterial pneumonia. Nat Med 14:275-281.
38. Zheng, Y., P. A. Valdez, D. M. Danilenko, Y. Hu, S. M. Sa, Q. Gong, A. R. Abbas, Z. Modrusan, N. Ghilardi, F. J. de Sauvage, and W. Ouyang. 2008. Interleukin-22 mediates early host defense against attaching and effacing bacterial pathogens. Nat Med 14:282-289.
39. Kudva, A., E. V. Scheller, K. M. Robinson, C. R. Crowe, S. M. Choi, S. R. Slight, S. A. Khader, P. J. Dubin, R. I. Enelow, J. K. Kolls, and J. F. Alcorn. Influenza A inhibits Th17-mediated host defense against bacterial pneumonia in mice. J Immunol 186:1666-1674.
40. Graham, A. C., K. D. Carr, A. N. Sieve, M. Indramohan, T. J. Break, and R. E. Berg. IL-22 production is regulated by IL-23 during Listeria monocytogenes infection but is not required for bacterial clearance or tissue protection. PLoS One 6:e17171.
41. Kagami, S., H. L. Rizzo, S. E. Kurtz, L. S. Miller, and A. Blauvelt. IL-23 and IL-17A, but not IL-12 and IL-22, are required for optimal skin host defense against Candida albicans. J Immunol 185:5453-5462.
42. Wilson, M. S., C. G. Feng, D. L. Barber, F. Yarovinsky, A. W. Cheever, A. Sher, M. Grigg, M. Collins, L. Fouser, and T. A. Wynn. Redundant and pathogenic roles for IL-22 in mycobacterial, protozoan, and helminth infections. J Immunol 184:4378-4390.
43. De Luca, A., T. Zelante, C. D'Angelo, S. Zagarella, F. Fallarino, A. Spreca, R. G. Iannitti, P. Bonifazi, J. C. Renauld, F. Bistoni, P. Puccetti, and L. Romani. IL-22 defines a novel immune pathway of antifungal resistance. Mucosal Immunol 3:361-373.
44. Sonnenberg, G. F., L. A. Monticelli, M. M. Elloso, L. A. Fouser, and D. Artis. CD4(+) lymphoid tissue-inducer cells promote innate immunity in the gut. Immunity 34:122-134.
45. Stange, J., M. R. Hepworth, S. Rausch, L. Zajic, A. A. Kuhl, C. Uyttenhove, J. C. Renauld, S. Hartmann, and R. Lucius. IL-22 mediates host defense against an intestinal intracellular parasite in the absence of IFN-gamma at the cost of Th17-driven immunopathology. J Immunol 188:2410-2418.
46. Munoz, M., M. M. Heimesaat, K. Danker, D. Struck, U. Lohmann, R. Plickert, S. Bereswill, A. Fischer, I. R. Dunay, K. Wolk, C. Loddenkemper, H. W. Krell, C. Libert, L. R. Lund, O. Frey, C. Holscher, Y. Iwakura, N. Ghilardi, W. Ouyang, T. Kamradt, R. Sabat, and O. Liesenfeld. 2009. Interleukin (IL)-23 mediates Toxoplasma gondii-induced immunopathology in the gut via matrixmetalloproteinase-2 and IL-22 but independent of IL-17. J Exp Med 206:3047-3059.
47. Arias, J. F., R. Nishihara, M. Bala, and K. Ikuta. High systemic levels of interleukin-10, interleukin-22 and C-reactive protein in Indian patients are associated with low in vitro replication of HIV-1 subtype C viruses. Retrovirology 7:15.
48. Biasin, M., M. Clerici, and L. Piacentini. Innate immunity in resistance to HIV infection. J Infect Dis 202 Suppl 3:S361-365.
49. Misse, D., H. Yssel, D. Trabattoni, C. Oblet, S. Lo Caputo, F. Mazzotta, J. Pene, J. P. Gonzalez, M. Clerici, and F. Veas. 2007. IL-22 participates in an innate anti-HIV-1 host-resistance network through acute-phase protein induction. J Immunol 178:407-415.
50. Dambacher, J., F. Beigel, K. Zitzmann, M. H. Heeg, B. Goke, H. M. Diepolder, C. J. Auernhammer, and S. Brand. 2008. The role of interleukin-22 in hepatitis C virus infection. Cytokine 41:209-216.
51. Pagliaccetti, N. E., E. N. Chu, C. R. Bolen, S. H. Kleinstein, and M. D. Robek. Lambda and alpha interferons inhibit hepatitis B virus replication through a common molecular mechanism but with different in vivo activities. Virology 401:197-206.
52. Robek, M. D., B. S. Boyd, and F. V. Chisari. 2005. Lambda interferon inhibits hepatitis B and C virus replication. J Virol 79:3851-3854.
53. Gad, H. H., C. Dellgren, O. J. Hamming, S. Vends, S. R. Paludan, and R. Hartmann. 2009. Interferon-lambda is functionally an interferon but structurally related to the interleukin-10 family. J Biol Chem 284:20869-20875.
54. Li, J., S. Hu, L. Zhou, L. Ye, X. Wang, J. Ho, and W. Ho. Interferon lambda inhibits herpes simplex virus type I infection of human astrocytes and neurons. Glia 59:58-67.
55. Levillayer, F., M. Mas, F. Levi-Acobas, M. Brahic, and J. F. Bureau. 2007. Interleukin 22 is a candidate gene for Tmevp3, a locus controlling Theiler's virus-induced neurological diseases. Genetics 176:1835-1844.
56. Guo, H., and D. J. Topham. Interleukin-22 (IL-22) production by pulmonary Natural Killer cells and the potential role of IL-22 during primary influenza virus infection. J Virol 84:7750-7759.
57. Monticelli, L. A., G. F. Sonnenberg, M. C. Abt, T. Alenghat, C. G. Ziegler, T. A. Doering, J. M. Angelosanto, B. J. Laidlaw, C. Y. Yang, T. Sathaliyawala, M. Kubota, D. Turner, J. M. Diamond, A. W. Goldrath, D. L. Farber, R. G. Collman, E. J. Wherry, and D. Artis. Innate lymphoid cells promote lung-tissue homeostasis after infection with influenza virus. Nat Immunol 12:1045-1054.
58. Guillot, L., R. Le Goffic, S. Bloch, N. Escriou, S. Akira, M. Chignard, and M. Si-Tahar. 2005. Involvement of toll-like receptor 3 in the immune response of lung epithelial cells to double-stranded RNA and influenza A virus. J Biol Chem 280:5571-5580.
59. Zemlickova, H., M. I. Crisostomo, M. C. Brandileone, T. Camou, E. Castaneda, A. Corso, G. Echaniz-Aviles, M. Pasztor, and A. Tomasz. 2005. Serotypes and clonal types of penicillin-susceptible streptococcus pneumoniae causing invasive disease in children in five Latin American countries. Microb Drug Resist 11:195-204.
60. Marques, J. M., A. Rial, N. Munoz, F. X. Pellay, L. Van Maele, H. Leger, T. Camou, J. C. Sirard, A. Benecke, and J. A. Chabalgoity. Protection against Streptococcus pneumoniae serotype 1 acute infection shows a signature of Th17- and IFN-gamma-mediated immunity. Immunobiology 217:420-429.
61. Munoz, N., L. Van Maele, J. M. Marques, A. Rial, J. C. Sirard, and J. A. Chabalgoity. Mucosal administration of flagellin protects mice from Streptococcus pneumoniae lung infection. Infect Immun 78:4226-4233.
62. Kreymborg, K., R. Etzensperger, L. Dumoutier, S. Haak, A. Rebollo, T. Buch, F. L. Heppner, J. C. Renauld, and B. Becher. 2007. IL-22 is expressed by Th17 cells in an IL-23-dependent fashion, but not required for the development of autoimmune encephalomyelitis. J Immunol 179:8098-8104.
63. Sparwasser, T., S. Gong, J. Y. Li, and G. Eberl. 2004. General method for the modification of different BAC types and the rapid generation of BAC transgenic mice. Genesis 38:39-50.
64. Lochner, M., L. Peduto, M. Cherrier, S. Sawa, F. Langa, R. Varona, D. Riethmacher, M. Si-Tahar, J. P. Di Santo, and G. Eberl. 2008. In vivo equilibrium of proinflammatory IL-17+ and regulatory IL-10+ Foxp3+ RORgamma t+ T cells. J Exp Med 205:1381-1393.
65. Paget, C., S. Ivanov, J. Fontaine, F. Blanc, M. Pichavant, J. Renneson, E. Bialecki, J. Pothlichet, C. Vendeville, G. Barba-Spaeth, M. R. Huerre, C. Faveeuw, M. Si-Tahar, and F. Trottein. Potential role of invariant NKT cells in the control of pulmonary inflammation and CD8+ T cell response during acute influenza A virus H3N2 pneumonia. J Immunol 186:5590-5602.
66. Belz, G. T., W. Xie, J. D. Altman, and P. C. Doherty. 2000. A previously unrecognized H-2D(b)-restricted peptide prominent in the primary influenza A virus-specific CD8(+) T-cell response is much less apparent following secondary challenge. J Virol 74:3486-3493.
67. Ivanov, S., J. Fontaine, C. Paget, E. Machofernandez, L. Van Maele, J. Renneson, I. Maillet, N. M. Wolf, A. Rial, H. Leger, B. Ryffel, B. Frisch, J. A. Chabalgoity, J. C. Sirard, A. Benecke, C. Faveeuw, and F. Trottein. Key role for respiratory CD103+ dendritic cells, IFN-gamma and IL-17 in protection against Streptococcus pneumoniae infection in response to alpha-galactosylceramide. J Infect Dis.
68. Reynders, A., N. Yessaad, T. P. Vu Manh, M. Dalod, A. Fenis, C. Aubry, G. Nikitas, B. Escaliere, J. C. Renauld, O. Dussurget, P. Cossart, M. Lecuit, E. Vivier, and E. Tomasello. Identity, regulation and in vivo function of gut NKp46+ RORgammat+ and NKp46+ RORgammat-lymphoid cells. Embo J30:2934-2947.
69. Taubenberger, J. K., and D. M. Morens. 2008. The pathology of influenza virus infections. Annu Rev Pathol 3:499-522.
70. McCullers, J. A. 2006. Insights into the interaction between influenza virus and pneumococcus. Clin Microbiol Rev 19:571-582.
71. van der Sluijs, K. F., T. van der Poll, R. Lutter, N. P. Juffermans, and M. J. Schultz. Bench-to-bedside review: bacterial pneumonia with influenza—pathogenesis and clinical implications. Crit Care 14:219.
72. Hartshorn, K. L. New look at an old problem: bacterial superinfection after influenza. Am J Pathol 176:536-539.
73. Ballinger, M. N., and T. J. Standiford. Postinfluenza bacterial pneumonia: host defenses gone awry. J Interferon Cytokine Res 30:643-652.
74. Snelgrove, R. J., A. Godlee, and T. Hussell. Airway immune homeostasis and implications for influenza-induced inflammation. Trends Immunol 32:328-334.
75. McCullers, J. A., J. L. McAuley, S. Browall, A. R. Iverson, K. L. Boyd, and B. Henriques Normark. Influenza enhances susceptibility to natural acquisition of and disease due to Streptococcus pneumoniae in ferrets. J Infect Dis 202:1287-1295.
76. Kash, J. C., K. A. Walters, A. S. Davis, A. Sandouk, L. M. Schwartzman, B. W. Jagger, D. S. Chertow, Q. Li, R. E. Kuestner, A. Ozinsky, and J. K. Taubenberger. Lethal synergism of 2009 pandemic H1N1 influenza virus and Streptococcus pneumoniae coinfection is associated with loss of murine lung repair responses. M Bio 2.
77. Koppe, U., N. Suttorp, and B. Opitz. Recognition of Streptococcus pneumoniae by the innate immune system. Cell Microbiol 14:460-466.
78. Kadioglu, A., and P. W. Andrew. 2004. The innate immune response to pneumococcal lung infection: the untold story. Trends Immunol 25:143-149.
79. Hamada, H., L. Garcia-Hernandez Mde, J. B. Reome, S. K. Misra, T. M. Strutt, K. K. McKinstry, A. M. Cooper, S. L. Swain, and R. W. Dutton. 2009. Tc17, a unique subset of CD8 T cells that can protect against lethal influenza challenge. J Immunol 182:3469-3481.
80. Crowe, C. R., K. Chen, D. A. Pociask, J. F. Alcorn, C. Krivich, R. I. Enelow, T. M. Ross, J. L. Witztum, and J. K. Kolls. 2009. Critical role of IL-17RA in immunopathology of influenza infection. J Immunol 183:5301-5310.
81. Morens, D. M., J. K. Taubenberger, and A. S. Fauci. 2008. Predominant role of bacterial pneumonia as a cause of death in pandemic influenza: implications for pandemic influenza preparedness. J Infect Dis 198:962-970.
82. Goulding, J., A. Godlee, S. Vekaria, M. Hilty, R. Snelgrove, and T. Hussell. Lowering the threshold of lung innate immune cell activation alters susceptibility to secondary bacterial superinfection. J Infect Dis 204:1086-1094.
83. Kumar P, Thakar M S, Ouyang W, Malarkannan S. IL-22 from conventional NK cells is epithelial regenerative and inflammation protective during influenza infection. Mucosal Immunol. 2012 Jun. 27.

1. A method for the prophylactic treatment of a bacterial superinfection post-influenza in a subject in need thereof comprising administering to the subject a therapeutically effective amount of an interleukin 22 (IL-22) polypeptide.

2. The method of claim 1 wherein the influenza infection is associated with Influenza virus A or B.

3. The method of claim 2 wherein the influenza infection is caused by an influenza virus A that is H1N1, H2N2, H3N2 or H5N1.

4. The method of claim 1 wherein the bacterial superinfection is selected from the group consisting of lower respiratory tract infections, middle ear infections and bacterial sinusitis.

5. The method of claim 1 wherein the bacterial superinfection may be mediated by at least one organism selected from the group consisting of Streptococcus pneumoniae; Staphylococcus aureus; Haemophilus influenza, Myoplasma species and Moraxella catarrhalis.

6. The method of claim 1 wherein the subject is selected from the group consisting of subjects who are at least 50 years old, subjects who reside in chronic care facilities, subjects who have chronic disorders of the pulmonary or cardiovascular system, subjects who required regular medical follow-up or hospitalization during the preceding year because of chronic metabolic diseases, renal dysfunction, hemoglobinopathies, or immunosuppression, children less than 14 years of age, patients between 6 months and 18 years of age who are receiving long-term aspirin therapy, and women who will be in the second or third trimester of pregnancy during the influenza season.

7. The method of claim 1 which is suitable for the prevention of bacterial superinfection post-influenza in subjects older than 1 year old and less than 14 years old;
subjects between the ages of 50 and 65, and adults who are older than 65 years of age.

8. The method of claim 1 wherein the subject has an IL-22 deficiency.

9. The method of claim 1 wherein the IL-22 polypeptide is administered to the patient in combination with an anti-bacterial agent.

10. The method of claim 13 wherein the antibiotic is selected from the group consisting of ceftriaxone, cefotaxime, vancomycin, meropenem, cefepime, ceftazidime, cefuroxime, nafcillin, oxacillin, ampicillin, ticarcillin, ticarcillin/clavulinic acid (Timentin), ampicillin/sulbactam (Unasyn), azithromycin, trimethoprim-sulfamethoxazole, clindamycin, ciprofloxacin, levofloxacin, synercid, amoxicillin, amoxicillin/clavulinic acid (Augmentin), cefuroxime, trimethoprim/sulfamethoxazole, azithromycin, clindamycin, dicloxacillin, ciprofloxacin, levofloxacin, cefixime, cefpodoxime, loracarbef, cefadroxil, cefabutin, cefdinir, and cephradine.

11. A method of testing whether an influenza infected patient is at risk of having a bacterial superinfection post-influenza which comprises the step of analyzing a biological sample from said patient for (i) detecting the presence of a mutation in the gene encoding for IL-22 and/or its associated promoter, and/or (ii) analyzing the expression of the gene encoding for IL-22.

12. The method of claim 11 wherein when the patient is considered at risk of having a bacterial superinfection is eligible for the prophylactic treatment of claim 1.

13. The method of claim 9, wherein said anti-bacterial agent is an antibiotic.
